(19) United States
(12) Patent Application Publication
(10) Pub. No.: US 2020/0405381 A1
(43) Pub. Date: Dec. 31, 2020

(54) SURFACE ABLATION DEVICE, SYSTEM AND METHOD

(71) Applicant: Covidien LP, Mansfield, MA (US)

(72) Inventor: Joe Sartor, Longmont, CO (US)

(21) Appl. No.: 16/908,054

(22) Filed: Jun. 22, 2020

Related U.S. Application Data
(60) Provisional application No. 62/867,355, filed on Jun. 27, 2019.

(51) Int. Cl. A61B 18/14; A61B 18/12; A61B 34/35; A61B 34/00

(52) U.S. Cl. CPC A61B 18/1482; A61B 18/1206; A61B 2018/00577; A61B 34/76; A61B 34/35

(57) ABSTRACT

An end effector assembly includes a base portion including a pivot assembly. A first portion of the pivot assembly is pivotally coupled to the distal end portion of the elongated shaft. A second portion of the pivot assembly is pivotally coupled to a distal portion of the push rod. The push rod moves the second portion of the pivot assembly between a proximal position and a distal position. An electrode substantially covers a distal surface of the base portion. The electrode receives electrosurgical energy from the electrosurgical generator and transmits the electrosurgical energy to treat tissue.

### CROSS-REFERENCE TO RELATED APPLICATIONS

This application claims priority to and the benefit of U.S. Provisional Application No. 62/867,355 filed Jun. 27, 2019. The disclosure of the foregoing application is hereby incorporated by reference in its entirety herein.

### BACKGROUND

### Technical Field

The present disclosure relates to ablation devices, systems, and methods for treating tissue. More specifically, the present disclosure relates to devices, systems and methods for surface ablation.

### Background of Related Art

In minimally-invasive surgical procedures, operations are carried out within an internal body cavity through small entrance openings in the body. The entrance openings may be natural passageways of the body or may be surgically created, for example, by making a small incision or hole into which an instrument is inserted.

Minimally-invasive surgical procedures may be used for treating internal body cavities, such as a wall of an abdominal cavity. Minimally-invasive surgical procedures may be performed by introducing an end effector of an electrosurgical device into an internal body cavity, contacting an electrode of the end effector to an abdominal wall and applying electrosurgical energy. However, controlling the surface area and depth to which the electrosurgical energy is applied from an electrode is important. The amount of electrosurgical energy applied by an electrode to a treatment tissue may vary based on an amount of energy applied. Additionally, the amount of surface area of the treatment tissue in contact with the electrode may also determine the amount of electrosurgical energy applied to the treatment tissue by the electrode.

During surgery it may be necessary to precisely effect tissue for necrosis, to prevent blood or serum loss or to treat diseased tissue. As an example, treating endometriosis on an abdominal wall or providing surface hemostasis when transecting organs (e.g., the liver) may be difficult when such treatments are performed in a minimally-invasive context. There is a need to treat both small and large surfaces though a small opening and at any instrument angle that is determined by the location of the minimally invasive entry port and the location of the target tissue.

### SUMMARY

In accordance with an aspect of the present disclosure, a surface ablation system includes an electrosurgical generator, a body, and an elongated shaft extending from the body. The elongated shaft includes a proximal portion coupled to the body and a distal end portion. The elongated shaft extends along a longitudinal direction. A push rod is connected with the body. The push rod extends along the longitudinal direction. The push rod moves between a retracted position and an extended position. An end effector is supported at the distal end portion of the elongated shaft. The end effector includes a base portion including a pivot assembly. A first portion of the pivot assembly is pivotally coupled to the distal end portion of the elongated shaft. A second portion of the pivot assembly is pivotally coupled to a distal portion of the push rod. The push rod moves the second portion of the pivot assembly between a proximal position and a distal position. An electrode substantially covers a distal surface of the base portion. The electrode receives electrosurgical energy from the electrosurgical generator and transmits the electrosurgical energy to treat tissue.

In some aspects, the electrode includes a toe portion and a heel portion. The heel portion is positioned proximal of the toe portion when the push rod is in the retracted position such that electrosurgical energy is provided to a first tissue treatment area to treat tissue by the toe portion. The heel portion substantially aligns with the toe portion when the push rod is in the extended position such that electrosurgical energy is provided to a second tissue treatment area to treat tissue by the heel and toe portions. The second tissue treatment area is larger than the first.

In some aspects, the electrode includes a toe portion and a heel portion. The heel portion is positioned proximal of the toe portion when the push rod is in the extended position such that electrosurgical energy is provided to a first tissue treatment area to treat tissue by the toe portion. The heel portion substantially aligns with the toe portion when the push rod is in the retracted position such that electrosurgical energy is provided to a second tissue treatment area to treat tissue by the heel and toe portions. The second tissue treatment area is larger than the first.

In some aspects, the electrode is an intertwined bipolar electrode.

In some aspects, the electrode is an intertwined bipolar electrode.

In some aspects, the first portion of the pivot assembly is rotatably coupled to the distal end portion of the elongated shaft by a pivot pin.

In some aspects, the heel portion defines a curved shape. The curved shape defined by the heel portion has substantially a same diameter as a curved shape defined by the elongated shaft.

In some aspects, the end effector defines a curved shape having a diameter equal to or less than a diameter of a curved shape defined by the elongated shaft.

In accordance with an aspect of the present disclosure, an end effector assembly includes a base portion including a pivot assembly. The pivot assembly is configured to allow movement of the end effector assembly between a proximal position and a distal position. An electrode substantially covers a distal surface of the base portion. The electrode receives electrosurgical energy from an electrosurgical generator and transmit the electrosurgical energy to treat tissue. The electrode includes a toe portion and a heel portion. The heel portion is positioned proximal of the toe portion when the end effector assembly is in the proximal position such that electrosurgical energy is provided to a first tissue treatment area to treat tissue by the toe portion. The heel portion substantially aligns with the toe portion when the end effector assembly is in the distal position such that electrosurgical energy is provided to a second tissue treatment area to treat tissue by the heel and toe portions. The second tissue treatment area is larger than the first tissue treatment area.

In accordance with an aspect of the present disclosure, a surface ablation method includes passing an end effector of a surface ablation device having an electrode into an internal body cavity through a natural orifice or incision. The method includes contacting a toe portion of the electrode against a first tissue treatment area to enable treatment of tissue within the first treatment area upon activation of the surface ablation device with electrosurgical energy. A push rod of the surface ablation device is advanced to bring a heel portion of the electrode into contact with a second tissue treatment area. The surface ablation device is activated to apply electrosurgical energy to the toe and heel portions of the electrode to enable treatment of tissue within the first and second tissue treatment areas.

In some aspects, the toe portion of the electrode defines a distal-most end of the end effector when the push rod is in the retracted position. The toe portion of the electrode is introduced through the aperture before the heel portion.

### BRIEF DESCRIPTION OF THE DRAWINGS

The accompanying drawings, which are incorporated in and constitute a part of this specification, illustrate embodiments of the present disclosure and, together with the detailed description below, serve to further explain the present disclosure, in which:

FIG. 1 is a side view of a surface ablation system including an end effector in accordance with the present disclosure;

FIG. 2 is an enlarged, side, perspective view of the end effector of FIG. 1 with a heel portion in a retracted position relative to a toe portion in accordance with the present disclosure;

FIG. 3 is an enlarged, rear perspective view of the end effector of FIG. 1 in accordance with the present disclosure;

FIG. 4A is an enlarged, side view of the end effector of FIG. 1 with the toe portion contacting a first tissue treatment area and the heel potion in a retracted position in accordance with the present disclosure; and

FIG. 4B is an enlarged, side view of the end effector of FIG. 1 with the toe portion contacting the first tissue treatment area and the heel portion in an extended position contacting a second tissue treatment area in accordance with the present disclosure.

### DETAILED DESCRIPTION

As used herein, the term “distal” refers to the portion that is being described which is further from a user, while the term “proximal” refers to the portion that is being described which is closer to a user. Further, to the extent consistent, any of the aspects and features detailed herein may be used in conjunction with any or all of the other aspects and features detailed herein.

As used herein, the terms parallel and perpendicular are understood to include relative configurations that are substantially parallel and substantially perpendicular up to about + or −10 degrees from true parallel and true perpendicular.

“About” or “approximately” as used herein may be inclusive of the stated value and means within an acceptable range of variation for the particular value as determined by one of ordinary skill in the art, considering the measurement in question and the error associated with measurement of the particular quantity (e.g., the limitations of the measurement system). For example, “about” may mean within one or more standard variations, or within ±30%, 20%, 10%, 5% of the stated value.

Descriptions of technical features or aspects of an exemplary embodiment of the present disclosure should typically be considered as available and applicable to other similar features or aspects in another exemplary embodiment of the present disclosure. Accordingly, technical features described herein according to one exemplary embodiment of the present disclosure may be applicable to other exemplary embodiments of the present disclosure, and thus duplicative descriptions may be omitted herein.

Exemplary embodiments of the present disclosure will be described more fully below (e.g., with reference to the accompanying drawings). Like reference numerals may refer to like elements throughout the specification and drawings.

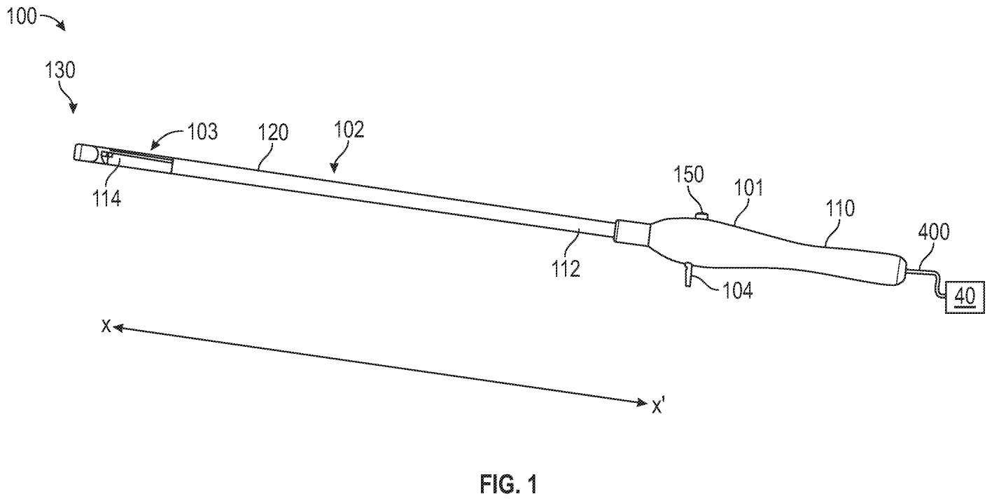

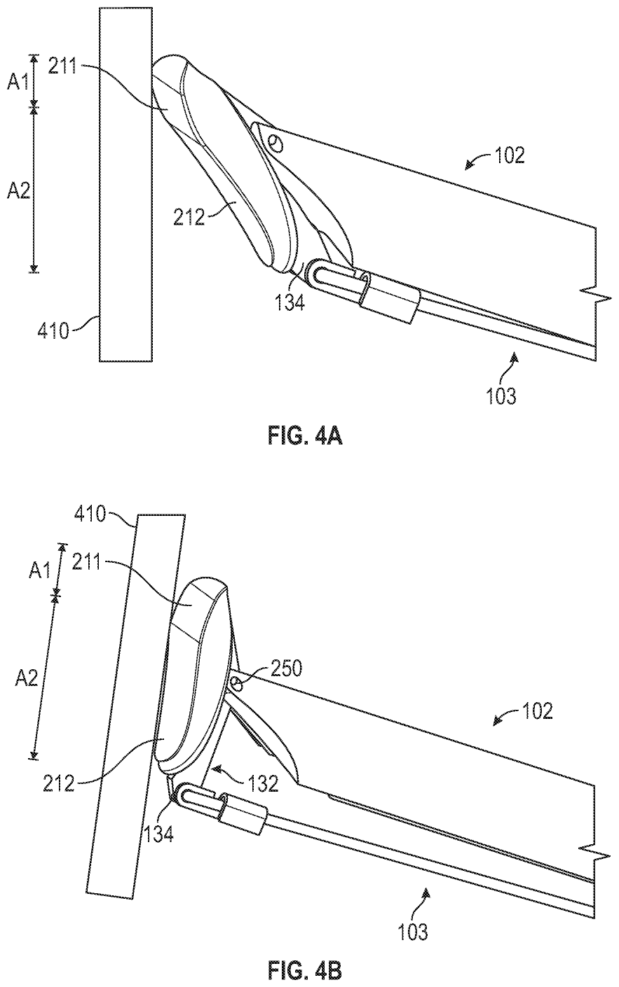

Referring to FIGS. 1-3, 4A and 4B, a surface ablation system 100 includes an electrosurgical generator 40, a body 101, a body handle 110, and an elongated shaft 102 extending from the body 101. The elongated shaft 102 includes a proximal portion 112 coupled to the body 101 and a distal portion 114. The elongated shaft 102 extends along a longitudinal direction (see, e.g., longitudinal direction X-X in FIG. 1). A push rod 103 is connected with the body 101 and extends along the longitudinal direction X-X. The push rod 103 is selectively movable via lever 104 disposed on body 101 between a retracted position (see, e.g., FIG. 4A) and an extended position (see, e.g., FIG. 4B).

An end effector 130 is supported at the distal portion 114 of the elongated shaft 102. The end effector 130 includes a base portion 131 including a pivot assembly 132. A first portion 133 of the pivot assembly 132 is pivotally coupled to the distal portion 114 of the elongated shaft 102. A second portion 134 of the pivot assembly 132 is pivotally coupled to a distal portion 214 of the push rod 103. The push rod 103 moves the second portion 134 of the pivot assembly 132 between a proximal position (see, e.g., FIG. 4A) and a distal position (see, e.g., FIG. 4B). An electrode 210 substantially covers a distal surface of the base portion 131. The base portion 301 provides both a relatively small and a relatively large treatment area with respect to a minimally-invasive access port. The electrode 210 receives electrosurgical energy from the electrosurgical generator 40 and transmits the electrosurgical energy to treat tissue (see, e.g., tissue 410 in FIGS. 4A and 4B). The electrosurgical generator 40 is connected with a distal portion of the body 101 by a cable 400 which conducts electrosurgical energy to the end effector 130. The electrode 210 includes a toe portion 211 and a heel portion 212. The heel portion 212 is positioned proximal of the toe portion 211 (relative to longitudinal axis X-X) when the push rod 103 is in the retracted position such that electrosurgical energy is provided to a first tissue treatment area (e.g., first area A1 in FIG. 4A) by the toe portion 211 (see, e.g., FIG. 4A) when the electrode 210 is selectively activated. The heel portion 212 substantially aligns with the toe portion 211 (relative to the longitudinal axis X-X) when the push rod 103 is in the extended position such that electrosurgical energy is provided to a second tissue treatment area (e.g., second area A2 in FIG. 4B) by the heel and toe portions 212 and 211 (see, e.g., FIG. 4B) when the electrode 210 is selectively activated. The second tissue treatment area is larger than the first tissue treatment area.

In some aspects, the position of the pivot assembly 132 may be distal of the toe portion 211, such that the toe portion 211 is advanced and retracted by the push rod 103.

In some aspects, the electrode 210 is an intertwined bipolar electrode (IBE) designed for surface ablation. A more detailed description of the IBE can be found in U.S. patent application Ser. No. 16/356,230, filed on Mar. 18, 2019, the entire disclosure of which is incorporated by reference herein. Briefly, IBE (i.e., end effector electrode 210) employs a series of intertwined flex circuits forming an end effector electrode 210 suitable for surface ablation in a predetermined target zone of treatment tissue. As applied here, the IBE has a shape corresponding with a shape of the base portion 131 to substantially and cover a surface of the base portion 131 facing away from the body 101.

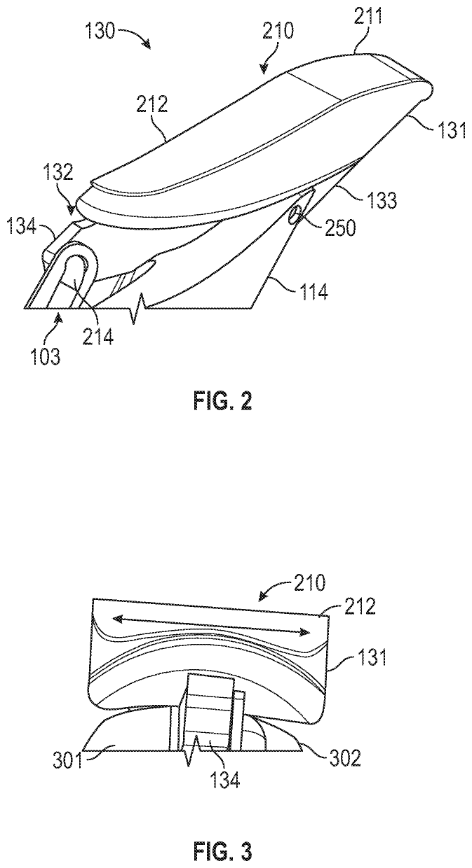

In some aspects, the first portion 133 of the pivot assembly 132 is rotatably coupled to the distal portion 114 of the elongated shaft 102 by a pivot pin 250 (see, e.g., FIG. 2). The pivot pin 250 extends through a hole (not shown) formed in the distal portion 114 of the elongated shaft 102. The distal portion 114 of elongated shaft 102 includes a first support arm 301 and a second support arm 302 (see, e.g., FIG. 3). The second portion 134 of the pivot assembly 132 may be positioned between the first support arm 301 and the second support arm 302.

In some aspects, the heel portion 212 defines a curved shape (see, e.g., FIG. 3). The curved shape defined by the heel portion 212 may have substantially a same diameter as a curved shape defined by the elongated shaft 102. In some aspects, the end effector 130 may define a curved shape having a diameter equal to or less than a diameter of the curved shape defined by the elongated shaft 102. In this instance, an incision or hole (e.g., having a circumference of about 5 mm, 8 mm, 10 mm, or 12 mm) that is typically formed for laparoscopic surgery to pass the elongated shaft 102 therethrough, would also be of sufficient size to pass the end effector 130 therethrough. That is, when the push rod 103 is in the retracted position and the heel portion 212 is correspondingly in the distal position, the overall circumference of the end effector 130 is substantially the same as the overall circumference of the elongated shaft 102. Thus, a minimally-sized hole is sufficient to pass the end effector 130 into a surgical cavity for surface ablation.

Moving the push rod 103 to the extended position after the end effector 130 is positioned in the surgical cavity advances the heel portion 212 into substantial alignment with the toe portion 211 (relative to the longitudinal axis X-X). Thus, the toe and heel portions 211 and 212 may each be contacted against a desired treatment area to apply electrosurgical energy thereto. The treatment area of tissue which electrosurgical energy is applied by both the toe and heel portions 211 and 212 of the electrode 210 is typically larger than the size of the hole or incision through which the end effector 130 initially passes. Thus, a relatively large treatment area may be treated by the end effector 130 while employing a relatively small hole or incision for introduction of the end effector 130.

In some aspects, the push rod 103 may be positioned outside the elongated shaft 102. Alternatively, the push rod 103 may be positioned in an internal space defined inside the elongated shaft 102. The push rod 103 may also be positioned outside the elongated shaft 102, but inside an interior space formed by an outer sleeve 120 positioned about the elongated shaft 102. In some aspects, the application of electrosurgical energy may be activated by depressing an activation button 150 disposed on the body 101.

In accordance with another aspect of the present disclosure, a method of ablating tissue is also disclosed. The method includes passing an end effector 130 of a surface ablation device having an electrode 210 into an internal body cavity through a natural orifice or incision. The method includes contacting a toe portion 211 of the electrode 210 against a first tissue treatment area (e.g., area A1) to enable treatment of tissue within the first treatment area upon activation of the surface ablation device with electrosurgical energy. A push rod 103 of the surface ablation device is advanced to bring a heel portion 212 of the electrode 210 into contact with a second tissue treatment area (e.g., area A2). The surface ablation device is activated to apply electrosurgical energy to the toe and heel portions 211 and 212 of the electrode 210 to enable treatment of tissue within the first and second tissue treatment areas.

In some aspects, the toe portion 211 of the electrode 210 may define a distal-most end of the end effector 130 when the push rod 103 is in the retracted position. In this configuration, the toe portion 211 of the electrode 210 may be introduced through the aperture (not shown) before the heel portion 212. Thus, a relatively small hole or incision may be sufficient for passing the end effector assembly 130 therethrough.

The various embodiments disclosed herein may also be configured to work with robotic surgical systems and what is commonly referred to as “Telesurgery.” Such systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A surface ablation system, comprising:
an electrosurgical generator;
a body;
an elongated shaft extending from the body, the elongated shaft including a proximal portion coupled to the body and a distal portion, the elongated shaft extending along a longitudinal direction;
a push rod connected with the body, the push rod extending along the longitudinal direction, and the push rod configured to move between a retracted position and an extended position; and
an end effector supported at the distal portion of the elongated shaft, the end effector including:
a base portion including a pivot assembly, a first portion of the pivot assembly pivotally coupled to the distal portion of the elongated shaft, and a second portion of the pivot assembly pivotally coupled to a distal portion of the push rod, the push rod configured to move the second portion of the pivot assembly between a proximal position and a distal position; and
an electrode substantially covering a distal surface of the base portion, the electrode configured to receive electrosurgical energy from the electrosurgical generator and transmit the electrosurgical energy to treat tissue.

2. The surface ablation system of claim 1, wherein the electrode includes a toe portion and a heel portion, the heel portion positioned proximal of the toe portion when the push rod is in the retracted position such that electrosurgical energy is provided to a first tissue treatment area to treat tissue by the toe portion, and wherein the heel portion substantially aligns with the toe portion when the push rod is in the extended position such that electrosurgical energy is provided to a second tissue treatment area to treat tissue by the heel and toe portions, the second tissue treatment area being larger than the first tissue treatment area.

3. The surface ablation system of claim 1, wherein the electrode includes a toe portion and a heel portion, the heel portion positioned proximal of the toe portion when the push rod is in the extended position such that electrosurgical energy is provided to a first tissue treatment area to treat tissue by the toe portion, and wherein the heel portion substantially aligns with the toe portion when the push rod is in the retracted position such that electrosurgical energy is provided to a second tissue treatment area to treat tissue by the heel and toe portions, the second tissue treatment area being larger than the first tissue treatment area.

4. The surface ablation system of claim 1, wherein the electrode is an intertwined bipolar electrode.

5. The surface ablation system of claim 1, wherein the first portion of the pivot assembly is rotatably coupled to the distal portion of the elongated shaft by a pivot pin.

6. The surface ablation system of claim 2, wherein the heel portion defines a curved shape.

7. The surface ablation system of claim 6, wherein the curved shape defined by the heel portion has substantially a same diameter as a curved shape defined by the elongated shaft.

8. The surface ablation system of claim 1, wherein the end effector defines a curved shape having a diameter equal to or less than a diameter of a curved shape defined by the elongated shaft.

9. An end effector assembly, comprising:
a base portion including a pivot assembly configured to allow movement of the end effector assembly between a proximal position and a distal position; and
an electrode substantially covering a distal surface of the base portion, the electrode adapted to receive electrosurgical energy from an electrosurgical generator and transmit the electrosurgical energy to treat tissue,
the electrode including a toe portion and a heel portion, the heel portion positioned proximal of the toe portion when the end effector assembly is disposed in the proximal position such that electrosurgical energy is provided to a first tissue treatment area to treat tissue by the toe portion, and wherein the heel portion substantially aligns with the toe portion when the end effector assembly is in the distal position such that electrosurgical energy is provided to a second tissue treatment area to treat tissue by the heel and toe portions, the second tissue treatment area being larger than the first tissue treatment area.

10. The end effector assembly of claim 9, wherein the electrode is an intertwined bipolar electrode.

11. The end effector assembly of claim 9, wherein the heel portion defines a curved shape.

12. The end effector assembly of claim 9, wherein a portion of the pivot assembly is coupled to a support arm at a distal portion of an elongated shaft.

13. The end effector assembly of claim 12, wherein the distal portion of the elongated shaft includes first and second support arms, and wherein the portion of the pivot assembly is positioned between the first and second support arms.

14. A method of ablating tissue, comprising:
passing an end effector of a surface ablation device having an electrode into an internal body cavity through a natural orifice or incision;
contacting a toe portion of the electrode against a first tissue treatment area to enable treatment of tissue within the first treatment area upon activation of the surface ablation device with electrosurgical energy; advancing a push rod of the surface ablation device to bring a heel portion of the electrode into contact with a second tissue treatment area; and
activating the surface ablation device to apply electrosurgical energy to the toe and heel portions of the electrode to enable treatment of tissue within the first and second tissue treatment areas.

15. The method of claim 14, wherein the electrode is an intertwined bipolar electrode.

16. The method of claim 14, wherein the heel portion defines a curved shape.

17. The method of claim 14, wherein the heel portion is positioned proximal of the toe portion when the push rod is in a retracted position, and wherein the heel portion is substantially aligned with the toe portion when the push rod is in an extended position.

18. The method of claim 14, wherein the toe portion of the electrode defines a distal-most end of the end effector when the push rod is in a retracted position.
